(19) United States
(12) Patent Application Publication
(10) Pub. No.: US 2024/0227512 A1
(43) Pub. Date: Jul. 11, 2024

(54) APPARATUS FOR PREVENTING CONTAMINATION OF VEHICLE AIR CONDITIONER

(71) Applicant: Proxihealthcare Inc., Ulsan (KR)

(72) Inventor: Young Wook Kim, Seoul (KR)

(21) Appl. No.: 18/539,284

(22) Filed: Dec. 14, 2023

Related U.S. Application Data
(63) Continuation of application No. PCT/KR2021/019116, filed on Dec. 15, 2021.

(30) Foreign Application Priority Data
Jun. 16, 2021 (KR) 10-2021-0077770

(51) Int. Cl. B60H 3/06; B60H 1/00

(52) U.S. Cl. CPC B60H 3/0633; B60H 1/00521

(57) ABSTRACT

The present invention relates to an apparatus for preventing contamination of a vehicle air conditioner, the apparatus being coupled to a vehicle air conditioner that comprises an evaporator for cooling down intaken air by means of a coolant, so as to prevent contamination of the vehicle air conditioner. The apparatus includes: an electrode part disposed on one surface of the evaporator and including a first electrode and a second electrode spaced apart from each other; and a signal supply configured to generate a driving signal by mixing an AC signal and a DC signal and supply the driving signal to the electrode part.

### CROSS REFERENCE TO RELATED APPLICATIONS

This application is a Bypass Continuation of International Application No. PCT/KR2021/019116, filed on Dec. 15, 2021, and claims priority from and the benefit of Korean Patent Application No. 10-2021-0077770, filed on Jun. 16, 2021 each of which is incorporated by reference for all purposes as if fully set forth herein.

### BACKGROUND

### Field

The present invention relates to an apparatus for preventing contamination in a car air conditioner by using a special microcurrent electromagnetic wave.

### Discussion of the Background

In general, air conditioners in cars are divided into ventilation systems and air conditioning systems, where a ventilation system is a device that keeps the air in a car comfortable by replacing polluted air in the car with fresh air from outside, or by circulating the air in the car and removing contaminants with filters, and an air conditioning system is a device that maintains the temperature in the car at a selected level.

Such a car air conditioner has the disadvantage that the surface of an evaporator is easily contaminated by condensate. In particular, the formation of fine dust and mold causes bacteria to proliferate, which reduces the efficiency of the air conditioner and degrades the air quality inside the car, such as clogged exhaust passages and bad smells.

### SUMMARY

An object of the present invention, which is made to solve the above-mentioned problem, is to provide an apparatus for preventing contamination in a car air conditioner that is capable of effectively preventing the car air conditioner from being contaminated by using an electromagnetic wave.

In addition, another object of the present invention is to provide an apparatus for preventing contamination in a car air conditioner that enhances the removal effect of a biofilm that causes contamination in the car air conditioner by using a driving signal generated by mixing an alternating current (AC) signal and a direct current (DC) signal.

According to an embodiment of the present invention, an apparatus for preventing contamination in a car air conditioner, which is coupled to the car air conditioner including an evaporator that cools inhaled air with a refrigerant, may include: an electrode part disposed on one surface of the evaporator and including a first electrode and a second electrode spaced apart from each other; and a signal supply configured to generate a driving signal by mixing an AC signal and a DC signal and supply the driving signal to the electrode part.

In addition, the electrode part may further include an electrode holder, and the electrode holder includes: a frame in which the first electrode and the second electrode are disposed; and at least one fixture configured to extend from the frame and provided with a fitting groove formed to be fitted into the evaporator.

In addition, the first electrode may have a shape in which a plurality of bars is arranged along a first direction, and the second electrode may have a shape in which a plurality of bars is arranged along a second direction intersecting the first direction.

In addition, the signal supply may be configured to modify at least one of the characteristics of the driving signal in response to an external input.

In addition, the characteristics of the driving signal may include an amplitude and a DC offset.

In addition, the signal supply may be configured to modify at least one of the characteristics of the driving signal according to an operating time of the car air conditioner.

In addition, the signal supply may be configured to generate a first driving signal and supply it to the electrode part during a period of operation of the car air conditioner, and generate a second driving signal and supply it to the electrode part when the operation of the air conditioner is stopped, and a magnitude of the second driving signal is larger than a magnitude of the first driving signal.

In addition, the apparatus may further include a resistance measuring device configured to measure the resistance of at least one of the first electrode and the second electrode, and the signal supply may be configured to modify at least one of an amplitude and a DC offset of the drive signal in response to a resistance value measured by the resistance measuring device.

Next, according to another embodiment of the present invention, an apparatus for preventing contamination in a car air conditioner, which is coupled to the car air conditioner including an air conditioner filter to filter out foreign matter in inhaled air and an evaporator to cool the inhaled air with a refrigerant, may include: an electrode part disposed on one surface of the air conditioner filter and including a first electrode and a second electrode spaced apart from each other; and a signal supply configured to generate a driving signal by mixing an AC signal and a DC signal and supply the driving signal to the electrode part.

Next, according to another embodiment of the present invention, an apparatus for preventing contamination in a car air conditioner, may include: an electrode holder; a first electrode and a second electrode fixedly installed in the electrode holder and spaced apart from each other; and a signal supply configured to generate a driving signal, which is a mixture of an AC signal and a DC signal, wherein the signal supply supplies the driving signal to the first electrode and supplies a ground voltage to the second electrode.

Next, according to another embodiment of the present invention, an apparatus for preventing contamination in a car air conditioner, which is coupled to the car air conditioner including an evaporator that cools inhaled air with a refrigerant, may include: an electrode part disposed on one surface of the evaporator and including a first electrode and a second electrode spaced apart from each other; and a signal supply configured to generate a driving signal by mixing an AC signal and a DC signal and supply the driving signal to the electrode part.

According to the present invention, it is possible to provide an apparatus for preventing contamination in the car air conditioner that is capable of effectively preventing the contamination in the car air conditioner from being contaminated by using the electromagnetic wave.

Furthermore, according to the present invention, it is possible to provide an apparatus for preventing contamination in the car air conditioner that enhances the removal effect of a biofilm that causes the contamination in the car air conditioner by using the driving signal generated by mixing the AC signal and the DC signal.

### BRIEF DESCRIPTION OF THE DRAWINGS

FIG. 1 is a diagram illustrating a schematic configuration of a car air conditioner.

FIG. 2 is a diagram illustrating an apparatus for preventing contamination in a car air conditioner according to an embodiment of the present invention.

FIG. 3 is a diagram illustrating that an electrode part is coupled to an evaporator according to an embodiment of the present invention.

FIG. 4 is an exploded view of the configuration shown in FIG. 3.

FIGS. 5A and 5B are diagrams explaining the removal effect of a biofilm by the driving signal generated by mixing an AC signal and a DC signal.

FIG. 6 is a diagram illustrating a signal supply module according to an embodiment of the present invention.

FIGS. 7A to 7C are diagrams illustrating the waveforms of signals according to an embodiment of the present invention.

FIG. 8 is a view showing a controller and a signal supply module according to an embodiment of the present invention.

FIG. 9 is a diagram illustrating a resistance measuring device, a controller, and a signal supply module according to an embodiment of the present invention.

FIG. 10 is a diagram illustrating that an electrode part is coupled to an air conditioner filter according to an embodiment of the present invention.

### DETAILED DESCRIPTION

In the following, embodiments related to the present invention are illustrated in the drawings and described in detail by way of a detailed description. However, the present invention is not limited to the embodiments disclosed below and may be implemented in various different forms. Also, it should be understood that all modifications, equivalents, or replacements thereof are included within the subject matter and scope of the present invention.

In describing elements of the present disclosure, terms such as first, second, A, B, (a), and (b) may be used. These terms are only used to distinguish one element from other elements, and the nature, sequence, or order of that element is not limited by the term. Further, it should be understood in this specification that if an element is described as being “connected”, “combined”, or “coupled” to/with any other element, the element may be directly connected, combined, or coupled to/with the other element, but another element may also be connected, combined, or coupled between both elements. In the case of being “connected”, “combined”, or “coupled”, it may be understood as being physically or electrically connected, combined, or coupled, but is also electrically “connected”, “combined”, or “coupled” as needed.

Terms such as “˜ unit”, “˜er”, “part”, and “˜ module” used in this specification refer to a unit that processes at least one particular function or operation, and may be implemented with hardware, software, or a combination thereof. In addition, terms such as “comprise”, “include”, and “have” used in this specification denote the presence of a stated element unless the relevant context clearly indicates otherwise, and do not exclude the presence of or a possibility of addition of one or more other elements.

In addition, it should be clarified that the division of the elements herein is merely based on the primary function performed by each element. That is, two or more elements to be described below may be combined into one element, or one element may be divided into two or more elements according to subdivided functions. It should also be noted that each element described below may, in addition to its primary function, perform some or all of the functions performed by other elements, and that a portion of the primary function of each element may be performed by other elements.

Hereinafter, an apparatus for preventing contamination in a car air conditioner according to an embodiment of the present invention will be described with reference to drawings related to embodiments of the present invention.

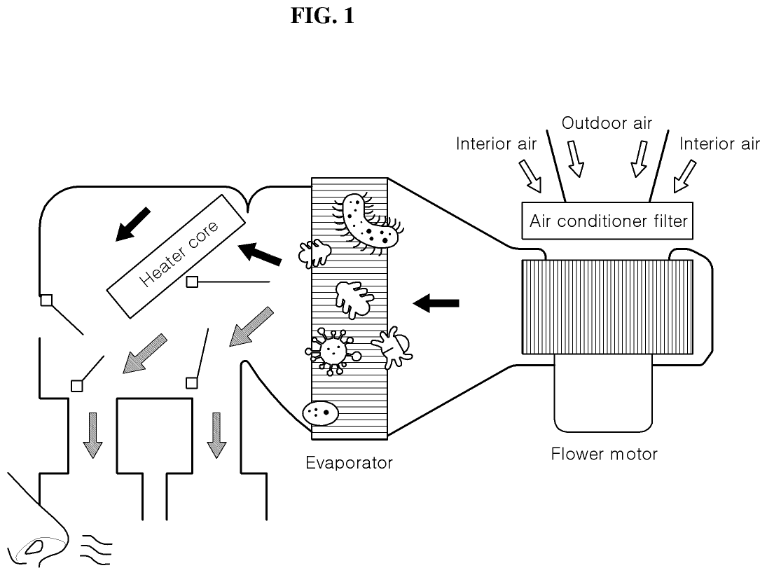

FIG. 1 is a diagram illustrating a schematic configuration of a car air conditioner.

Referring to FIG. 1, a car air conditioner, which is intended to regulate and maintain an interior temperature at a comfortable temperature to create a more pleasant and comfortable interior environment for a driver and passengers, includes a blower for inhaling outdoor air (outside air) or interior air (inside air), an air conditioner filter for filtering out foreign matter in the air inhaled by the blower, an evaporator for cooling the inhaled air with a refrigerant, and a heater core for heating the inhaled air with heat of a coolant.

However, with the passage of time, various foreign substances and dust adhere to the air conditioner filter, the evaporator, and the heater core, through which the air inhaled by the blower passes, providing conditions for various bacteria and mold to grow, and thus causing the driver and passengers to breathe in air contaminated with unpleasant odors and various bacteria. Especially in summer, when the temperature is high, various bacteria and molds actively proliferate, causing unpleasant odors and respiratory problems.

Accordingly, an apparatus for preventing contamination in an car air conditioner according to an embodiment of the present invention aims to provide an electromagnetic wave specialized for removing a biofilm that causes contamination to an internal components of the car air conditioner, such as the blower, the evaporator, the air conditioner filter, the heater core, and the like, in order to purify the air supplied from the car air conditioner to the interior of the car and to remove odors caused by the car air conditioner.

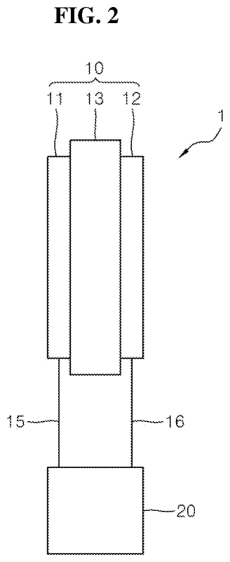

FIG. 2 is a diagram illustrating an apparatus for preventing contamination in a car air conditioner according to an embodiment of the present invention.

Referring to FIG. 2, an apparatus 1 for preventing contamination in a car air conditioner according to an embodiment of the present invention may include an electrode part 10 and a signal supply module 20.

The electrode part 10 may include a first electrode 11, a second electrode 12, and an electrode holder 13.

The first electrode 11 may be disposed on one side of the electrode holder 13, and may be supplied with a driving signal to provide electromagnetic waves corresponding to the driving signal.

That is, the first electrode 11 may emit the electromagnetic waves to the outside based on the electrical energy of the driving signal, and these electromagnetic waves may suppress the formation of a biofilm on the components of the car air conditioner, such as the evaporator, the air conditioner filter, etc., and remove the formed biofilm.

The second electrode 12 may be disposed on the other side of the electrode holder 13, and may be spaced apart from the first electrode 11. For example, when the first electrode 11 is set as a positive electrode, the second electrode 12 may be set as a negative electrode or ground electrode.

The first electrode 11 and the second electrode 12 may be formed of a material such as, but not limited to, copper, brass, aluminum, conductive polymer, conductive silicon, or stainless steel.

The signal supply module 20 may be electrically connected to the first electrode 11 and the second electrode 12 via separate wirings 15 and 16. For example, the signal supply module 20 may supply the driving signal to the first electrode 11 via the first wiring 15 and supply a ground voltage to the second electrode 12 via the second wiring 16.

The apparatus 1 for preventing contamination in a car air conditioner according to an embodiment of the present invention may be coupled to and operated in the car air conditioner such as that shown in FIG. 1, and in particular, the apparatus 1 may be coupled to at least one of an evaporator, an air conditioner filter, and a heater core in the car air conditioner which is provided with the electrode part 10.

The apparatus 1 for preventing contamination in the car air conditioner may operate together for the duration of the operation of the car air conditioner. Furthermore, the apparatus 1 for preventing contamination in the car air conditioner may be operable even during a period of stoppage of the car air conditioner, for example, for a predetermined period of time after the operation of the car air conditioner is stopped.

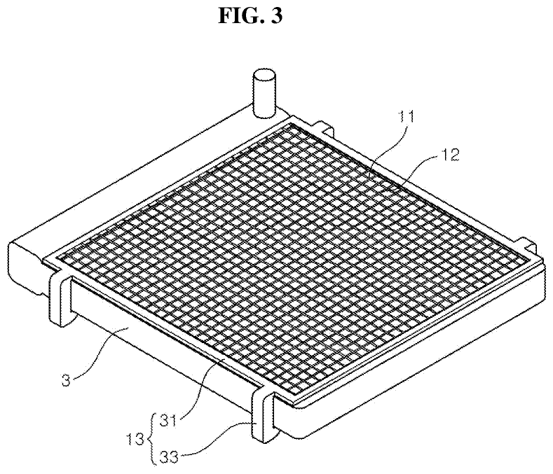

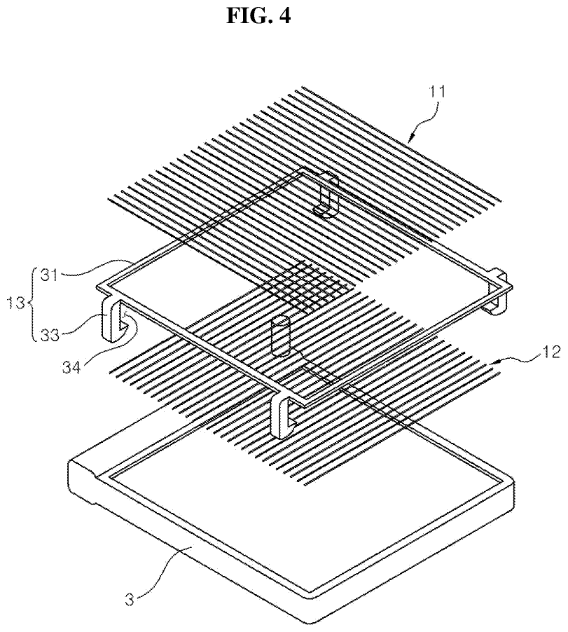

FIG. 3 is a diagram illustrating that an electrode part is coupled to an evaporator according to an embodiment of the present invention, and FIG. 4 is an exploded view of the configuration shown in FIG. 3.

Referring to FIGS. 3 and 4, the electrode part 10 according to an embodiment of the present invention is located on one surface of the evaporator 3, which is one of the components provided in the car air conditioner, so that the evaporator 3 can be prevented from being contaminated.

As described with reference to FIG. 2, the electrode part 10 includes the first electrode 11, the second electrode 12, and the electrode holder 13, wherein the first electrode 11 may have a shape in which a plurality of bars is arranged along a first direction.

The first electrode 11 may further include a separate connection wire (not shown) electrically connecting each of the plurality of bars, or alternatively, the first wiring 15 may be connected to each of the plurality of bars.

The second electrode 12 may have a shape in which a plurality of bars is arranged along a second direction intersecting the first direction. For example, the first direction may be set in a direction orthogonal to the second direction.

Accordingly, the first electrode 11 and the second electrode 12 located in the electrode holder 13 may have a mesh shape. Moreover, the arrangement of the first electrode 11 and the second electrode 12 to intersect each other enables smooth generation of an electromagnetic wave that suppresses and eliminates the formation of a biofilm.

The second electrode 12 may further include a separate connection wire (not shown) electrically connecting each of the plurality of bars, or alternatively, a second wiring 16 may be connected to each of the plurality of bars.

Meanwhile, the shape of the first electrode 11 and the second electrode 12 may not be limited to that shown in FIGS. 3 and 4, and may include at least one opening to allow air to pass through, but the shape may be variously changed.

The electrode holder 13 may include a frame 31 and at least one fixture 33.

The frame 31 may be formed in a shape corresponding to the shape of the evaporator 3, for example, a rectangular shape.

The first electrode 11 and the second electrode 12 may be disposed on the frame 31, and the first electrode 11 and the second electrode 12 may be attached to or inserted into the frame 31 to be fixed.

The fixture 33 may be formed to extend from the frame 31 and may have a fitting groove 34 formed to be fitted in the evaporator 3. Accordingly, it is possible for the fixture 33 to be fixedly coupled to a side portion of the evaporator 3 by means of the fitting groove 34.

Although FIGS. 3 and 4 illustrate the case where the first electrode 11 and the second electrode 12 are coupled to the evaporator 3 through the electrode holder 13, the first electrode 11 and the second electrode 12 may be manufactured integrally with the evaporator 3.

Furthermore, although the first wiring 15, the second wiring 16, and the signal supply module 20 are not shown in FIGS. 3 and 4, it should be noted that the first electrode 11 may receive the driving signal from the signal supply module 20 via the first wiring 15, and the second electrode 12 may receive the ground voltage from the signal supply module 20 via the second wiring 16.

In particular, the signal supply module 20 according to an embodiment of the present invention may generate the driving signal by mixing an alternating current (AC) signal and a direct current (DC) signal and supply it to the first electrode 11.

Accordingly, the driving signal may include both AC component and DC component, and synergistic effects and resonance may occur by simultaneously applying the AC component and DC component, thereby increasing the removal effect of a biofilm that causes mold and bacteria.

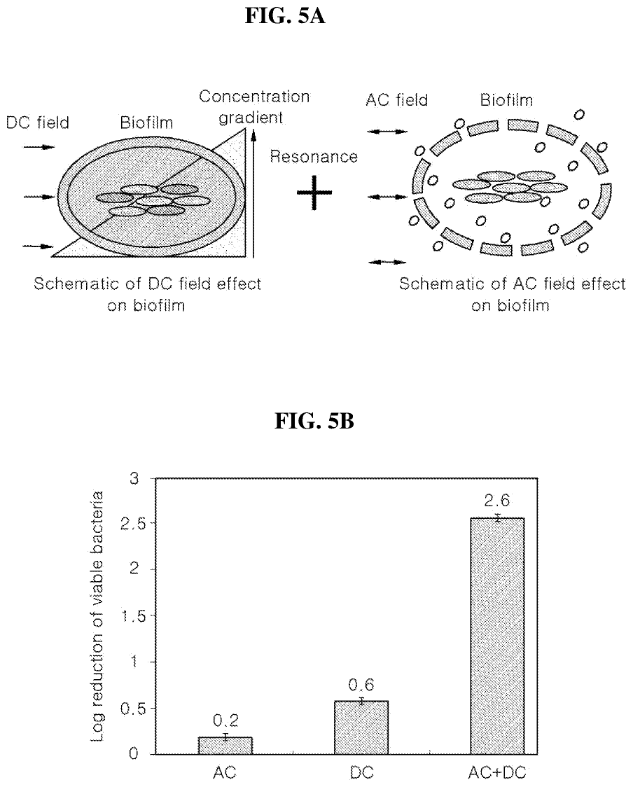

FIGS. 5a and 5b are diagrams explaining the removal effect of a biofilm by the driving signal generated by mixing an AC signal and a DC signal.

Referring to FIG. 5a, an electric field induced by the DC component of the driving signal may increase the structural stress on the biofilm by inducing an imbalance in the locally distributed charge, and an electric field induced by the AC component of the driving signal may increase the permeability to the external protector by generating specific vibrations.

The synergistic effect resulting from these AC component and DC component may be observed from FIG. 5b. Specifically, compared to the biofilm removal effect when the electric field induced by the AC component and the electric field induced by the DC voltage are provided separately, it can be seen that the biofilm removal effect is significantly superior when the electric field induced by the AC component and the electric field induced by the DC voltage are provided simultaneously in an overlapping manner.

Since the electric field induced by the DC component and the electric field induced by the AC component in response to the driving signal supplied from the signal supply module 20 according to an embodiment of the present invention may be provided simultaneously by the electrode 11, the above-described enhanced biofilm removal effect may be achieved.

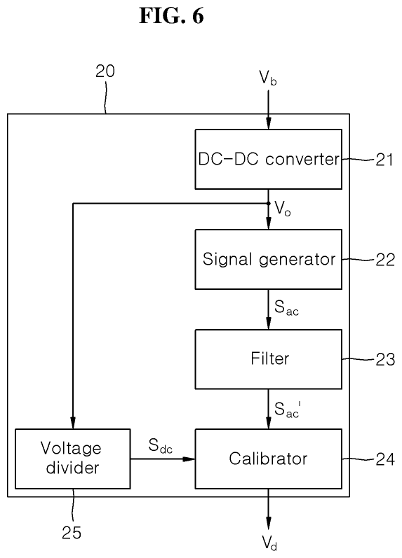

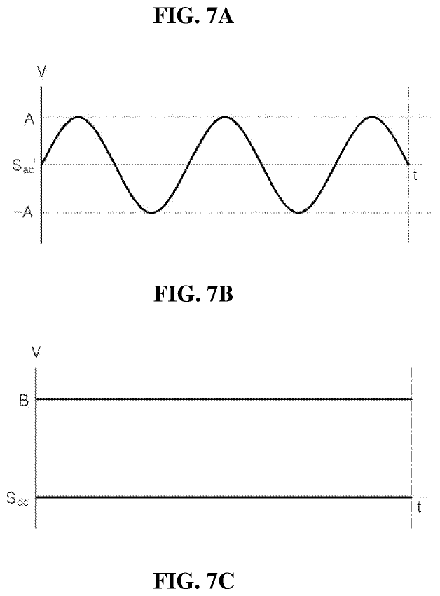

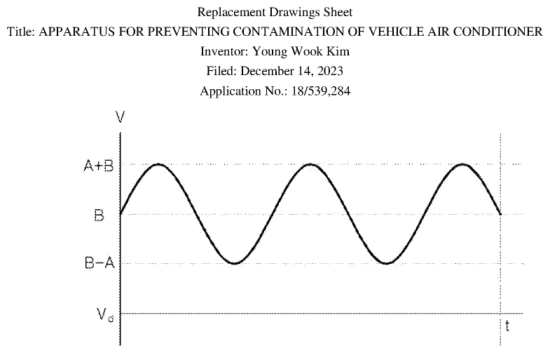

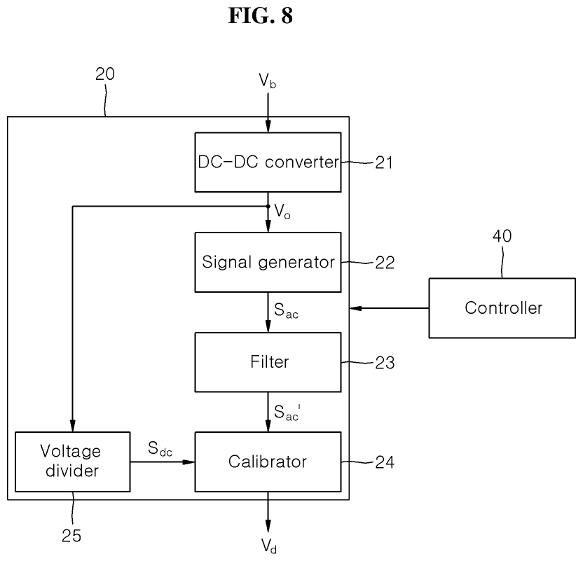

FIG. 6 is a diagram illustrating the signal supply module according to an embodiment of the present invention, and FIGS. 7a to 7c are diagrams illustrating the waveforms of signals according to an embodiment of the present invention. In particular, FIG. 7a shows a filtered AC signal Sac′, FIG. 7b shows a DC signal Sdc, and FIG. 7c shows the driving signal Vd generated by mixing the filtered AC signal Sac′ and the DC signal Sdc.

Referring to FIG. 6, the signal supply module 20 according to an embodiment of the present invention may include a DC-DC converter 21, a signal generator 22, a filter 23, and a calibrator 24, and may additionally include a voltage divider 25.

The DC-DC converter 21 may receive an external voltage Vb, convert the external voltage Vb into an output voltage Vo of a predetermined level, and output it.

The signal generator 22 may operate based on a voltage supplied from the DC-DC converter 21, and may generate an AC signal Sac with a predetermined frequency using the output voltage Vo from the DC-DC converter 21.

The signal generator 22 may be implemented using a known configuration, such as an oscillator, a function generator, or the like, capable of generating an AC signal.

For example, a frequency in the range of 1 KHz to 1000 MHz may be set for the AC signal Sac. This is because when the AC signal Sac is set to a low frequency below 1 KHz, the effect of removing the biofilm will be reduced, and even when the AC signal Sac is set to an ultra-high frequency above 1000 MHz, the effect of removing the biofilm will also be reduced. Meanwhile, the frequency of the AC signal Sac may be set to a frequency in the range of 5 MHz to 15 MHz suitable for biofilm removal.

In addition, the amplitude of the AC signal Sac may be set in the range of, but not limited to, 0.1 mv to 10 V, which is suitable for the removal of the biofilm.

The filter 23 may perform a filtering operation on the AC signal Sac generated by the signal generator 22. For example, the filter 23 may include a low-pass filter and may convert the AC signal Sac in a sawtooth waveform into the AC signal Sac′ in a sine waveform. However, the type of the filter 23 is not limited thereto, and various types of filters may be employed depending on the design structure.

The calibrator 24 may generate the driving signal Vd by mixing the DC signal Sdc with the AC signal Sac′ supplied through the filter 23. For example, the calibrator 24 may be implemented with an operating amplifier capable of summing (or superimposing) the AC signal Sac′ and the DC signal Sdc, but is not limited thereto.

Consequently, an offset corresponding to the DC signal Sdc may be generated in the AC signal Sac′, and thus the driving signal Vd containing both AC component and DC component may be generated.

Since the driving signal Vd includes all the characteristics of the AC signal Sac, the driving signal Vd may be set at a frequency in the range of 1 KHz to 1000 MHz and may also be set at a frequency in the range of 5 MHz to 15 MHz which is more suitable for the biofilm removal. In addition, the amplitude of the driving signal Vd may be set to in the range of 0.1 mV to 10V.

Referring to FIG. 7a, the calibrator 440 may receive the AC signal Sac′ having an amplitude of A volt (V) from the filter 23, and superimpose the DC signal Sdc of B volt (V) as shown in FIG. 7b on the AC signal Sac′, thereby generating a final driving signal Vd as shown in FIG. 7c.

In this case, the voltage value of the DC signal Sdc may be set to be equal to or greater than the amplitude of the AC signal Sac′. Accordingly, the voltage value of the driving signal Vd may be set to 0 or more.

Ultimately, the DC offset value of the driving signal Vd may be set to be equal to or greater than the amplitude of the driving signal Vd.

If the DC offset value of the driving signal Vd is less than the amplitude value of the driving signal Vd, it results in an interval where the voltage of the driving signal Vd has a negative value and the voltage in the interval has a negative value, causing a loss of electrical energy to occur.

However, when the DC offset value of the driving signal Vd is set equal to or greater than the amplitude of the driving signal Vd as in the embodiment of the present invention, it is possible to minimize the loss of electrical energy because the voltage of the driving signal Vd is always zero or greater.

Meanwhile, the DC signal Sdc may be generated by the voltage divider 25. For example, the voltage divider 25 may receive the output voltage Vo from the DC-DC converter 21, and perform voltage division on the output voltage Vo to generate the DC signal Sdc.

The voltage divider 25 may comprise, a resistor string for dividing the output voltage Vo, but is not limited thereto.

When the output voltage Vo of the DC-DC converter 21 is suitable to be used directly to generate the driving signal Vd, the corresponding output voltage Vo may serve as the DC signal Sdc. In this case, the voltage divider 25 may be omitted and the output voltage Vo of the DC-DC converter 21 may be inputted to the calibrator 24.

According to an embodiment of the present invention, the signal supply module 20 may modify a characteristic of the driving signal Vd depending on whether the car air conditioner is operating, and the characteristic of the driving signal Vd may include an amplitude and a DC offset of the driving signal Vd.

For example, the signal supply module 20 may control in such a manner that the magnitude of the driving signal Vd supplied to the first electrode 11 after the operation of the car air conditioner is stopped is larger than the magnitude of the driving signal Vd supplied to the first electrode 11 during the period of operation of the car air conditioner. Here, the magnitude of the driving signal Vd may mean the root mean square (RMS).

More specifically, the signal supply module 20 may generate a first driving signal and supply it to the first electrode 11 during a period of operation of the car air conditioner, and generate a second driving signal and supply it to the first electrode 11 when the operation of the car air conditioner is stopped, wherein the characteristic of the first driving signal and the characteristic of the second driving signal may be different.

In this case, the signal supply module 20 may control the second driving signal to be larger than the first driving signal by adjusting the magnitude of at least one of an amplitude and a DC offset of the first driving signal, and adjusting the magnitude of at least one of an amplitude and a DC offset of the second driving signal.

Further, the signal supply module 20 may modify the characteristic of the driving signal Vd according to the operating time of the car air conditioner. For example, it may be controlled to increase the magnitude of the driving signal Vd supplied to the first electrode 11 as the operating time of the car air conditioner increases.

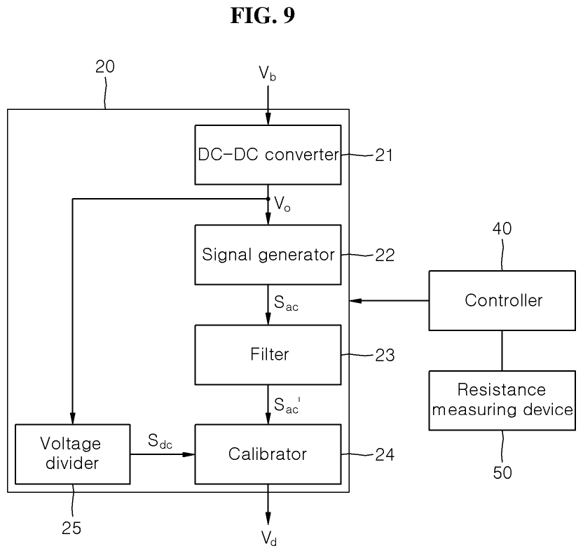

FIG. 8 is a diagram illustrating a controller and the signal supply module according to an embodiment of the present invention.

Referring to FIG. 8, the signal supply module 20 according to an embodiment of the present invention may modify at least one of the characteristics of the driving signal Vd in response to an external input.

For this purpose, a controller 40 may further be installed for controlling the signal supply module 20 in response to the external input (e.g., the user input).

In other words, by adjusting at least one of the amplitude and DC offset of the driving signal Vd, the user may set an optimal driving signal Vd for suppressing and/or eliminating the formation of the biofilm in the car air conditioner, and this enables the biofilm to be managed taking into account the characteristic for the situation-specific.

In this case, the user's input method for controlling the characteristic of the driving signal Vd may be set in various ways. For example, the user may control the signal supply module 20 through a separate terminal (not shown) or a separate input device (not shown), which may be provided in the apparatus 1 for preventing contamination in the car air conditioner.

When setting information for the driving signal Vd is inputted by the user, the controller 40 may control the signal supply module 20 to provide the driving signal Vd having amplitude and DC offset value corresponding to the inputted setting information.

The controller 40 may modify the amplitude of the AC signal Sac by controlling the signal generator 22. In addition, the controller 40 may adjust the voltage value of the DC signal Sdc by controlling the DC-DC converter 21 and/or the voltage divider 25. Therefore, the characteristic of the driving signal Vd may finally be modified.

In this case, the controller 40 may control the voltage divider 25 to set the voltage value of the DC signal Sdc to be equal to or greater than the amplitude of the AC signal Sac′, and thus the voltage value of the driving signal Vd may be set to be equal to or greater than zero.

FIG. 9 is a diagram illustrating a resistance measuring device, the controller, and the signal supply module according to an embodiment of the present invention.

Referring to FIG. 9, a resistance measuring device 50 may be further installed to measure the resistance of at least one of the first electrode 11 and the second electrode 12.

The resistance of the first electrode 11 and the second electrode 12 may be changed depending on the extent to which a biofilm occurs, and the controller 40 may control the characteristic of the driving signal Vd in response to the resistance value measured by the resistance measuring device 50.

In other words, the extent to which a biofilm occurs may be detected by the change in the resistance of the first electrode 11 and the second electrode 12, and at least one of the amplitude and the DC offset of the driving signal Vd may be adjusted to reflect the same, thereby increasing the effect of suppressing or eliminating the biofilm formation.

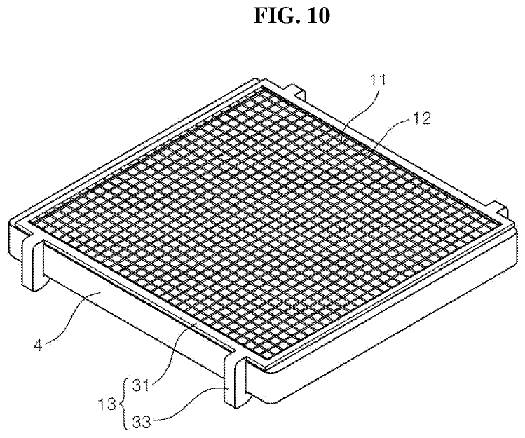

FIG. 10 is a diagram illustrating that an electrode part is coupled to an air conditioner filter according to an embodiment of the present invention. In describing an arrangement in which the electrode part is coupled to the air conditioner filter, the same reference numerals are assigned to the same components as in the embodiments described with reference to FIGS. 1 to 9, and any overlapping contents with the above-described embodiments are omitted while focusing on the differences.

Referring to FIG. 10, the electrode part 10 according to an embodiment of the present invention may be disposed on one surface of the air conditioner filter 4, which is one of the components provided in the car air conditioner.

The electrode part 10 may receive the driving signal Vd from the signal supply module 20 connected via the first wiring 15 and the second wiring 16 to provide an electromagnetic wave specialized for removing the biofilm to the air conditioner filter 4, thereby preventing the air conditioner filter 4 from being contaminated.

Although, in FIGS. 2, 3, 4, and 10, the evaporator 3 or the air conditioner filter 4 is shown to be a separate component from the apparatus 1 for preventing contamination in the car air conditioner according to an embodiment of the present invention, the present invention is not limited thereto. For example, the evaporator 3 or the air conditioner filter 4 may be incorporated in the apparatus for preventing contamination in the car air conditioner.

Furthermore, not only car air conditioners but also conventional air conditioners equipped with the evaporator 3 will be able to effectively remove the biofilm that causes the contamination in the air conditioner by employing the configuration described above.

Those skilled in the art to which the present invention pertains will understand that the present invention may be embodied in other specific forms without changing its technical idea or essential features. Therefore, the embodiments described above should be understood in all respects as illustrative and not restrictive. The scope of the present invention is defined by the following claims rather than by the above detailed description, and all changes or modifications derived from the claims and their equivalents should be construed as being within in the scope of the present invention.

1. An apparatus for preventing contamination in a car air conditioner, which is coupled to the car air conditioner including an evaporator that cools inhaled air with a refrigerant, the apparatus comprising:
an electrode part disposed on one surface of the evaporator and including a first electrode and a second electrode spaced apart from each other; and
a signal supply configured to generate a driving signal by mixing an AC signal and a DC signal and supply the driving signal to the electrode part.

2. The apparatus of claim 1, wherein the electrode part further includes an electrode holder, and wherein the electrode holder includes:
a frame in which the first electrode and the second electrode are disposed; and
at least one fixture configured to extend from the frame and provided with a fitting groove formed to be fitted into the evaporator.

3. The apparatus of claim 1, wherein the first electrode has a shape in which a plurality of bars is arranged along a first direction, and the second electrode has a shape in which a plurality of bars is arranged along a second direction intersecting the first direction.

4. The apparatus of claim 1, wherein the signal supply is configured to modify at least one of the characteristics of the driving signal in response to an external input.

5. The apparatus of claim 4, wherein the characteristics of the driving signal include an amplitude and a DC offset.

6. The apparatus of claim 5, wherein the signal supply is configured to modify at least one of the characteristics of the driving signal according to an operating time of the car air conditioner.

7. The apparatus of claim 1, wherein the signal supply is configured to generate a first driving signal and supply it to the electrode part during a period of operation of the car air conditioner, and generate a second driving signal and supply it to the electrode part when the operation of the air conditioner is stopped, and
a magnitude of the second driving signal is larger than a magnitude of the first driving signal.

8. The apparatus of claim 1, further comprising:
a resistance measuring device configured to measure the resistance of at least one of the first electrode and the second electrode, and
wherein the signal supply is configured to modify at least one of an amplitude and a DC offset of the drive signal in response to a resistance value measured by the resistance measuring device.

9. An apparatus for preventing contamination in a car air conditioner, which is coupled to the car air conditioner including an air conditioner filter to filter out foreign matter in inhaled air and an evaporator to cool the inhaled air with a refrigerant, the apparatus comprising:
an electrode part disposed on one surface of the air conditioner filter and including a first electrode and a second electrode spaced apart from each other; and
a signal supply configured to generate a driving signal by mixing an AC signal and a DC signal and supply the driving signal to the electrode part.

10. An apparatus for preventing contamination in a car air conditioner, the apparatus comprising:
an electrode holder;
a first electrode and a second electrode spaced apart from each other and fixedly installed in the electrode holder; and
a signal supply configured to generate a driving signal, which is a mixture of an AC signal and a DC signal, wherein the signal supply supplies the driving signal to the first electrode and supplies a ground voltage to the second electrode.

11. An apparatus for preventing contamination in a car air conditioner, which is coupled to the car air conditioner including an evaporator that cools inhaled air with a refrigerant, the apparatus comprising:
an electrode part disposed on one surface of the evaporator and including a first electrode and a second electrode spaced apart from each other; and
a signal supply configured to generate a driving signal by mixing an AC signal and a DC signal and supply the driving signal to the electrode part.
